(19) United States
(12) Patent Application Publication
(10) Pub. No.: US 2011/0217269 A1
(43) Pub. Date: Sep. 8, 2011

(54) METHOD OF FOSTERING THE HEALING OF SKIN WOUNDS USING CELLS OF THE ROOT SHEATH, COMPOSITION AND PREPARATION METHOD

(75) Inventor: Wolfgang RICHTER, Freising (DE)

(73) Assignee: EURODERM GMBH, Leipzig (DE)

(21) Appl. No.: 13/035,389

(22) Filed: Feb. 25, 2011

Related U.S. Application Data
(60) Provisional application No. 61/313,139, filed on Mar. 12, 2010.

(30) Foreign Application Priority Data
Feb. 26, 2010 (DE) 102010009572.9

(51) Int. Cl. A61K 35/36; C12N 11/16; A61P 17/02; C12N 5/00

(52) U.S. Cl. 424/93.7; 435/174; 435/325; 435/378

(57) ABSTRACT

The present invention relates to a method for promoting the healing of skin wounds or the re-establishment of the function of the skin and/or its skin appendices of a second skin area, comprising the step of: applying cells obtained from hair root sheaths of a first skin area of a donor onto a second skin area of a host. It further relates to cells, a preparation, the use of cells as well as a method for preparing a preparation.

### CROSS-REFERENCE TO RELATED APPLICATIONS

The present application claims priority under 35 U.S.C. §119 of German Patent Application No. 10 2010 009 572.9, filed on Feb. 26, 2010 and claims the benefit under 35 U.S.C. §119(e) of U.S. Provisional Application No. 61/313,139, filed Mar. 12, 2010. The disclosures of these applications are expressly incorporated by reference herein in their entireties.

The present invention relates to a method for promoting the healing of skin wounds and/or the re-establishment of the function of the skin and/or of its skin appendices according to the preamble of claim 1. It further relates to cells, in particular melanocyte precursor cells and/or keratinocyte precursor cells and/or mesenchymal precursor cells, according to claim 12, a preparation according to claim 15, the use of cells according to claim 16 as well as a method for preparing a preparation according to claim 19.

The use of melanocyte precursor cells and/or keratinocyte precursor cells for increasing or enhancing a pigmentation of specific skin areas is known, i.a. from PCT/EP 2008/007684 of the applicant of the present invention.

One object according to the invention is to provide further uses of body cells.

This object of the present invention is achieved by a method according to claim 1. Additionally, cells according to claim 12, a preparation comprising cells according to claim 15, the use of cells according to claim 16 as well as a method for preparing a preparation according to claim 19 are proposed.

According to claim 1, a method for promoting the healing of skin wounds of a second skin area and/or for promoting the re-establishment of the function of the skin and/or of its skin appendices is proposed. The method comprises applying cells obtained from hair root sheaths of a first skin area of a donor onto a second skin area of a recipient or host.

According to claim 12, cells of hair root sheaths of a first skin area of a donor are proposed that are or have been obtained for the purpose of being used in or on a second skin area for promoting the healing of wounds and/or the re-establishment of the function of the skin and/or of its skin appendices of the second skin area of a host.

According to claim 15, a preparation comprising the cells according to the invention is proposed.

According to claim 16, the use of cells obtained from hair root sheaths of a first skin area of a donor for producing a preparation for promoting the healing of wounds and/or the re-establishment of the function of the skin and/or of its skin appendices of a second skin area of a host is proposed.

According to claim 19, a method for producing a preparation, in particular a suspension, comprising cells, in particular melanocyte precursor cells and/or keratinocyte precursor cells and/or mesenchymal precursor cells, is proposed.

Advantageous developments are subject-matter of the dependent claims and embodiments. In the following, the terms “may be” or “can be” or “may have” or “can have”, respectively, etc. shall be understood as a synonym for “preferably is” or “preferably has” etc. and shall illustrate one embodiment according to the present invention.

The method according to the invention of claim 1 in some embodiments according to the invention serves a cosmetic purpose only, such as for increasing the pigmentation or for inhibiting hypertrophic scar tissue of the second skin area. In some embodiments according to the invention, the method according to the invention serves for fulfilling aesthetic demands of persons having been treated by means of the method. The method according to the invention of claim 1 in some embodiments according to the invention serves a non-therapeutic and non-surgical purpose.

Embodiments according to the present invention may comprise one or more of the following features.

In certain embodiments of the method according to the invention, the cells used are melanocyte precursor cells and/or keratinocyte precursor cells and/or mesenchymal precursor cells (hereafter referred to as: “precursor cells”) or comprise such cells in a cell aggregation or in a united cell structure or in a group or mixture. In some embodiments, the said cells are present in a cell mixture that can be obtained when the cells that have been obtained from the hair root sheath are not divided with respect to their origin, their function or their character or nature. If, however, the cells are divided after they have been obtained, in the sense of the present invention, a greater effect may be achieved after their application when using primarily melanocyte precursor cells and/or keratinocyte precursor cells and/or mesenchymal precursor cells.

If, in the following, melanocyte precursor cells or stem cells and/or keratinocyte precursor cells or stem cells are referred to this also applies without restrictions to mesenchymal precursor cells and stem cells even if this is not explicitly stated.

The keratinocyte precursor cells may be applied alternatively or additionally to the melanocyte precursor cells.

Applying the keratinocyte precursor cells may be done at the same time as performing the step of applying the melanocyte precursor cells. However, the keratinocyte and the melanocyte precursor cells may be applied at different points of time as well.

A combined and, when indicated, simultaneous use of melanocyte precursor cells and keratinocyte precursor cells may advantageously result in a promoted growth of the melanocyte precursor cells and the keratinocyte precursor cells after their simultaneous application onto the second skin area.

The applicant of the present method attributes or ascribes this to chemical, biochemical and/or biological interactions between the precursor cell types mentioned. The applicant observed that this advantage already occurs if the natural preparation ratio of keratinocyte precursor cells and melanocyte precursor cells such as, e.g., present in the hair root sheaths of the first skin area, is kept in the mixture of the cells that are applied onto the second skin area.

The application of cells—for example, of melanocyte precursor cells or stem cells and/or of keratinocyte precursor cells or stem cells—of hair root sheaths, in particular of epithelial hair root sheaths, of a first skin area onto a second skin area serves to promote the healing of skin wounds and/or to re-establish of the function of the skin and/or of its skin appendices of the second skin area or has this purpose. Applying the cells may thereby advantageously contribute to stimulating the growth, the differentiation and/or the function of the (skin) cells as well as of skin appendices such as hair and cells that are present in skin layers.

Promoting the healing of wounds and/or the re-establishment of the function of the skin and/or of its skin appendices of a second skin area of a host in the sense of the present invention comprises in some embodiments according to the invention promoting or aiding the primary or secondary healing of wounds of the second skin area or has this purpose. This may include (re-) epithelisation of the wound.

Promoting the healing of wounds and/or the re-establishment of the function of the skin and/or its skin appendices of a second skin area of a host in the sense of the present invention comprises in certain embodiments according to the invention reducing or inhibiting the development or occurrence of hypertrophic scar tissue or of keloid, e.g., during the wound healing of skin of a second skin area of the body surface or has this purpose.

Promoting the healing of wounds and/or the re-establishment of the function of the skin and/or of its skin appendices of a second skin area of a host in the sense of the present invention comprises in some embodiments according to the invention enhancing or promoting the hair-growth of a scar or of skin of the second skin area or has this purpose.

As compared to the application of, for example, interfollicular melanocytes, applying cells of the hair root sheath has the advantage of a comparably unlimited availability. Whereas obtaining melanocytes generally has to be done using skin extraction and is thus only suited for enhancing the wound healing or establishment of the function of a limited skin area—and has, moreover, to be done surgically at the extraction site—cells of the hair root sheath such as, e.g., melanocyte precursor cells and/or keratinocyte precursor cells may be obtained from the hair root sheaths in a comparably simple manner and in high quantity. The application of such cells thus implies their advantageously high availability.

In certain embodiments of the methods according to the invention, the cells that are used according with those methods are present in a suspension or in a sediment.

In certain embodiments of the present invention, the application of the cells is performed by simply brushing, spraying, dabbing, or the like. In some embodiments, the application is done non-invasively. In certain embodiments, it is done non-surgically. In some or all embodiments, application may be done without using physician or medical expertise.

Applying the cells or precursor cells may, e.g., by performed by using a suspension comprising 10²-10⁹ cells/ml, particularly 10⁵-10⁷ cells/ml, for example, by means of a syringe or a spray or in a biocompatible non-woven material.

The application can be done using a biocompatible solution (e.g., PBS) or by means of a biocompatible support (e.g., hyaluronan, collagen). Thereby, the cells may be used as vital cells or, e.g., as cells inhibited in their growth by using mitomycin C or radiation or as cell extracts (such as, e.g., lyophilisates, sonicates). Media conditioned with these cells may be used for this purpose as well.

The application of cells or precursor cells may be done by simply depositing the cells onto the skin. The cells may be fixed onto the second skin area by means of, e.g., a fibrin adhesive and may be secured by means of an occlusive or occlusion dressing. However, every other appropriate form of application, e.g., integrating the cells in biological or synthetic matrices, is also possible according to the invention.

In one embodiment of the method according to the invention, the cells have been obtained from hair root sheaths of the first skin area by means of plucking hairs out of or from the vital skin of the donor or by means of picking or plucking or otherwise obtaining hairs from or out of a present skin biopsy of the donor. Plucking the hairs in certain embodiments according to the present invention represents the only aspect of dividing and/or obtaining the cells of first skin area. In those embodiments, it is thus exclusively plucked. It is particularly not cut, punched, or the like.

The cells mentioned-above can thus advantageously be obtained repeatedly in a simple way. The process of obtaining the cells prior to their application may thus be performed in a comparably simple manner free of pain. Furthermore, this process does not include the risk of complications, in particular no or no significant risk of infection. In particular, no germs or pathogens are transferred when solely plucking the hairs or their hair root sheaths whereas said germs or pathogens are typically transferred in case of blood contact.

When using cells of the hair root sheath, the cells may, for example, be obtained by picking scalp hair, in particular anagen hair, in particular hair of the capillitium, which as mentioned above has another advantage as compared to the use of cells of other origin, in particular of interfollicular melanocytes.

“Obtaining” in the sense of the application in certain embodiments according to the invention refers to separating or releasing the cells, for example, the melanocyte precursor cells and/or the keratinocyte precursor cells, from the first skin area. According to the invention, it may also comprise releasing the cells from the first skin area. This release may, for example, comprise simply picking or plucking the hairs, in particular anagen hairs, of the scalp hair. In other embodiments according to the invention, obtaining explicitly does not include dividing the cells from the first skin area.

In the sense of the application, in some embodiments, obtaining refers to dividing the cells, optionally the preparation as well, e.g., by means of trypsin; or comprises such a division and/or preparation.

According to the invention, besides separating the cells from the first area—or alternatively hereto—, “obtaining” cells from hair root sheaths may comprise isolating the cells from the hair root sheaths, in particular from the epithelial hair root sheaths, as well.

The step of “obtaining” may also comprise one step or a plurality of steps by means of which the obtained cells are prepared for their application. This may be done by preparing a cell suspension.

The cell suspension, in particular a melanocyte precursor cell suspension and/or a keratinocyte precursor cell suspension may be prepared after an in vitro-propagation of the cells contained therein. In some embodiments, cultivation is included. In other embodiments, cultivation is not included.

However, the cells and/or the cell suspension may also be prepared directly, i.e., without any further cultivation or growth cultivation of the cells for the purpose of cultivating, differentiating or maturating the cells therein.

In some embodiments of the method according to the invention, the cells are applied without cell growth cultivation.

In certain embodiments according to the invention, obtaining comprises releasing the cells from the skin or scalp regardless of the skin being vital skin or a skin biopsy.

The suspension may contain biocompatible substances such as PBS and/or a biocompatible support such as, for example, hyaluronan or collagen.

In a further embodiment of the method according to the invention, the second skin area is prepared for receiving the cells. Such preparation allows for growing the cells applied onto the second skin area in a particularly effective way.

Preparing the second skin area may, for example, comprise releasing the epidermis or parts thereof from the second skin area. The latter is, for example, possible by using dermabrasio or superficial laser application accompanied by the advantages associated therewith and known to a person skilled in the art.

In certain embodiments according to the invention, preparation comprises applying an appropriate solution. An example of this is a fibrinogen solution preparing the second skin area for the subsequent reception of the cells and for a better adherence of the cells and a specific cell growth at the second skin area.

In certain embodiments of the method according to the invention, the second skin area is not and/or not in the course of the method according to the invention prepared for receiving the cells. The latter, e.g., applies if the cells or the precursor cells are applied onto an existing wound.

In some embodiments of the method according to the invention, the cells applied onto the second skin area are stimulated, e.g., for an accelerated pigment formation or for an accelerated maturation or differentiation.

Such a stimulation may be effected using UV radiation. The UV radiation activates the transferred cells and may, e.g., be achieved by using broad band UV, narrow band UV, PUVA or excimer laser radiation.

Stimulating may advantageously result in pigmentation as well as the establishment or formation of the epidermal pigmentation unit. It may result in an accelerated pigment formation.

Stimulating by using, for example, ultraviolet radiation may be done once or repeatedly. Thereby, radiation should advantageously be lower than the erythem limit, respectively. For example, radiating twice a week may result in a desired pigmentation of the second skin area. Radiating more or less frequently is also possible.

The applicant's examinations have shown that stimulation by means of radiation can also further promote wound healing. This was able to be shown after one UV stimulation.

The cells can be derived from one donor and can be re-applied to the donor again.

In some embodiments according to the invention, donor and host are identical. The first skin area and the second skin area are thus skin areas of one and the same individual. In such a case, efforts relating to typing or matching due to genetic differences between donor and host may advantageously be omitted or reduced. Additionally, the risk of transferring—for example, infections—from the donor to the host may be eliminated.

However, the cells can also be derived from a donor and can be applied to a host other than the donor. Thereby, it is irrelevant if donor and host are human or animal. A transfer between animal and human or vice versa is encompassed by the invention as well.

According to the invention, “melanocyte precursor cells” are to be understood as both autologous and allogenic and xenogenic melanocyte precursor cells or stem cells, respectively; “keratinocyte precursor cells” are to be understood as both autologous and allogenic and xenogenic keratinocyte precursor cells or stem cells, respectively; “mesenchymal precursor cells” are to be understood as both autologous and allogenic and xenogenic mesenchymal precursor cells or stem cells, respectively.

The object according to the invention is further achieved by means of cells, in particular melanocyte precursor cells and/or keratinocyte precursor cells and/or mesenchymal precursor cells, according to the features of claim 12. Advantageous developments are hereby in turn subject-matter of respective sub-claims.

As the same advantages that can be achieved by means of the method according to the invention described above may be achieved by means of the cells according to the invention, reference is made here to the discussion above in order to avoid repetition.

The cells according to the invention have been obtained from hair root sheaths of a first skin area. The cells according to the invention are thus present outside the body.

The cells according to the invention are, in some embodiments according to the invention, suited and provided for being used in or on a second skin area for promoting the healing of wounds and/or the re-establishment of the function of the skin and/or of its skin appendices of the second skin area.

In certain embodiments, the cells according to the invention have been obtained by means of picking or plucking hairs out of the vital skin of a donor or by means of plucking or otherwise obtaining hairs from a present skin biopsy of the donor.

In some embodiments, the cells have been obtained and are provided for their use without being or having been cultivated in a cell growth cultivation.

In certain embodiments, the cells are not or will not be changed or altered, in particularly not genetically altered.

The object according to the invention is further achieved by means of a preparation having the features of claim 15.

As the same advantages that can be achieved by means of the method according to the invention described above may be achieved by means of the preparation according to the invention, reference is made here to the discussion above in order to avoid repetitions.

The preparation according to the invention comprises cells according to the invention, for example, melanocyte precursor cells and/or keratinocyte precursor cells and/or mesenchymal precursor cells.

Furthermore, the object according to the invention is also achieved by means of a method having the features of claim 16 or of claim 19. The advantages achievable by means of this method are the same advantages that can be achieved by means of the method described above for which reason reference is made to the discussion above in order to avoid repetitions.

Thereby, the cells were able to be obtained by means of plucking hairs out of the vital skin of the donor or by means of plucking or otherwise obtaining hairs out of a present skin biopsy of the donor.

In one embodiment of the method according to the invention, the cells have been obtained and are provided for use without being or having been cultivated in a cell growth cultivation.

According to claim 19, a method for preparing a preparation is proposed. In some embodiments thereof, the preparation is a suspension. In certain embodiments, the preparation is provided and suited for use in any one of the methods described above.

The method according to the invention for preparing a preparation, in particular a suspension, in some embodiments comprises at least enzymatically detaching the cells—or precursor cells—regardless of whether the cells are melanocyte precursor cells and/or keratinocyte precursor cells and/or mesenchymal precursor cells—from the hair root sheath of an extracted hair. The enzymatic detachment may, for example, be done using a trypsin/EDTA solution.

EDTA may be present as a liquid in form of a clear, colorless, odorless solution, prepared according to Ph. Eur. (European pharmacopoeia in the current edition), having a pH of 5.42 to 6.04 and an osmolarity of 331-368 mOsm/kg such as is, for example, available from the company Biochrom AG, Germany under the article number L2113 in a 100 ml-glass flask.

The trypsin/EDTA solution can be 0.8%. The solution particularly effects the detachment of epithelial cells from the hair sheath. The detachment is preferably carried out at 37° C. However, higher temperatures, at which the viability of the cells can still be ensured, or lower temperatures, at which the activity of trypsin can still be ensured, are possible as well.

For the enzymatic detachment, an incubation in trypsin, 0.1% to 10%, in particular between 0.5% and 4%, in PBS of 1-50, in particular of 15-30 min can be carried out.

PBS may be obtained by the company BioConcept, Switzerland, in a 500 ml-flask in liquid form having a pH of 7.3±0.2 and an osmolarity of 285±10 in form of PBS without Ca/Mg having the article number 3-05F29 (PBS1) or in form of PBS comprising Ca/Mg having the article number 8-05F00 (PBS2) as a sterile, colorless and clear liquid that can be stored at room temperature. Such PBS may be suited for preparing an M solution.

The particular advantage of the enzymatic detachment is that, due to the possible use of enzymes, an adverse effect to the cells treated and/or the change or alteration thereof, i. a., a genetic change, can be avoided. Thus, enzymatic detachment is particularly gentle on the cells to be obtained.

Additionally, the method for preparing the suspension in some embodiments according to the invention—alternatively or additionally and independently from each other, respectively—comprises the following steps: a) stopping the enzymatic detachment by, e.g., adding human serum; b) centrifuging the suspension in order to obtain a sediment, c) re-suspending the cellulous sediment in a thrombin solution allowing for an immediate fixation of the cells applied in a thin layer for their application on fibrinogen that has been applied previously and thus enabling a homogenous not too occlusive application to every body region.

TissueCol-DuoS 2 ml Immuno can be used as the adhesive protein solution, for example prepared by the company Baxter AG, Austria under the article number B1332020110614 and available by the company Baxter Deutschland GmbH, Hyland Immuno Division, Germany. Such a biological two-component adhesive consists of 2 ready-to-use syringes comprising 2 ml adhesive protein solution with fibrinogen and 2 ml thrombin solution, respectively. After mixing the two components, solidification or hardening of the adhesive may be effected in seconds to minutes.

In some embodiments, the method comprises—independently from each other, respectively—the following steps: transferring the cells or the suspension or the sediment into a biocompatible solution, introducing or inserting the cells or the suspension or the sediment into a biocompatible support and/or preparing a cell extract.

In the following, exemplary embodiments are specified in detail.

Thus, in a first exemplary embodiment, a preparation has been prepared. For this, different solutions have been prepared that would theoretically be sufficient for four patients and that can variably be adapted for each individual case.

A dispase solution was prepared from 20 ml dispase and 40 ml PBS1 and was resuspended in 250 ml-culture medium flasks. Thereafter, 4 aliquots of 15 ml each were transferred into 50 ml-tubes for hair plucking.

PBS1 was aliquoted in 4×8 ml each for transferring the hair follicles into 50 ml-tubes.

For the trypsin solution, 4 ml trypsin solution (e.g., TS solution from Sigma), 2 ml EDTA (1.0% from Biochrom) and 4 ml PBS1 were mixed and aliquoted in 15 ml-tubes with 2 ml each.

The stopping solution was prepared by mixing 135 ml PBS2 with 15 ml human serum and resuspending the mixture in 250 ml-culture medium flasks. Thereafter, the solution was aliquoted in 50 ml-tubes with 30 ml each.

For the thrombin solution, 2 ml thrombin (TissueCol-DuoS) were mixed with 11.3 ml PBS2 (with Ca/Mg) and resuspended in 15 ml-tubes. For transportation, 2 ml were drawn into 2 ml-syringes comprising a sterile cannula (size 1) each, the air bubbles were removed, the cannula was provided with a protective cover again, the syringes were packed in adhesive bags and were sent in a non-frozen state together with a thermal pack.

The fibrinogen solution was prepared by mixing 2 ml fibrinogen (TissueCol-DuoS) with 6 ml PBS1. For transportation, 2 ml were drawn into 2 ml-syringes comprising a sterile cannula (size 1) each, the air bubbles were removed, the cannula was provided with a protective cover again, the syringes were packed in adhesive bags and were sent in a non-frozen state together with dry ice.

Per patient and/or treatment, the following devices or apparatuses and/or consumables or expendables may be used or optionally be sent together with a treatment set or kit or be prepared or provided for the treatment: pipettor and charging cable, sterile metal forceps, a 90 ml-petri dish, pipettes (10 pipettes with 10 ml/5 pipettes with 2 ml), a cellular sieve, 1-2 cryo-tubes; a 50 ml-tube containing 15 ml dispase solution, a 50 ml-tube containing 8 ml PBS1, a 15 ml-tube containing 2 ml trypsin solution, a 50 ml-tube 30 ml PBS2/human serum, a 50 ml-tube (for the cellular sieve), four 15 ml-tubes (for centrifugation); a syringe (comprising a cannula) containing 2 ml fibrinogen solution, a syringe (comprising a cannula) containing 2 ml thrombin solution.

At first, a cell suspension was prepared. Therefor, a fibrinogen solution (syringe) was thawed at room temperature. A dispase solution for hair plucking (15 ml) was transferred into a 90 ml-petri dish. Thereafter, the main part of the hair (dead hair material) was cut from about 250 hair follicles (sufficient for treating an area of about 20 to 30 cm²) that are already present in a plucked state. The hair follicles were transferred into the 90 ml-petri dish.

The cut hair follicles were transferred into a 50 ml-tube containing 8 ml PBS1 by means of a sterile forceps. Then, 2 ml trypsin solution was added. Attention should be paid that all hair follicles immersed into the solution.

The tube was heated (e.g., by means of a thermal block/water bath) at about 37° C. The trypsin treatment was performed for about 25-30 min under occasional shaking/resuspending. By adding 30 ml PBS2/serum, trypsin was deactivated.

A mechanical detachment of the ORS cells (cells of the outer root sheath; ORS=outer root sheath) was obtained by thoroughly pipetting (at least 30 times), e.g., by means of a 10 ml-pipette.

Then, the suspension was transferred through a cellular sieve (pore size 70 μm) into a 50 ml-tube in order to remove dead cell material/cell aggregates.

The suspension (40 ml) was distributed to four 15 ml-tubes. The cells were centrifuged and the supernatant was poured or pipetted away. The thrombin solution (2 ml) was added from the syringe to the cell pellet and the cells were resuspended by using a 2 ml-pipette. Thereby, the cells of the four tubes were collected.

It should be noted that in some embodiments of the method according to the invention centrifugation is not provided. In those embodiments, the method can be performed without using centrifugation. This may advantageously minimize the technical effort required for performing the method.

Subsequently, the cell suspension was transferred into a cryo-tube and then drawn into the thrombin syringe.

For treating skin wounds, 2 ml fibrinogen solution were applied onto the patients' wound area. Then, the thrombin cell suspension was applied. The wound was bandaged in the usual manner. The dressing was firstly changed after 5 days, respectively.

For treating scar tissue such as hypertrophic scars as well as keloid formation, the treated patients have been prepared in the following way. After local anesthesia of the wound, the scar tissue was ablated using dermabrasio or an erbium YAG laser. Then, the method according to the invention started with applying the cell suspension and the fibrin adhesive (optionally) by means of a syringe or a spraying device. The areas treated were covered with a plastic dressing. The dressing was changed after 5 to 7 days.

The treatment of a patient in which promoting wound healing, hair growth and avoiding the keloid formation in the area of the second skin area was observed is described exemplarily as follows:

The patient, male, 29 years old had abdominal surgery in 1991. After the operation, keloid had formed in the scar tissue. This had been treated for years with different conventional creams without any cosmetic improvement.

Yet, a one-time treatment using the method according to the invention in 2009 resulted—within only 4 days—in a complete epithelisation of the acute wound that has been generated by means of dermabrasio prior to applying the method according to the invention. In the course of the following months until now, keloid formation has not been observed; there was no keloid formation taking place. However, pigmentation adapted to the surrounding skin areas has been observed; furthermore, hair growth on the treated acute wound has been observed. In doing so, melanocyte precursor cells were applied to the patient.

When applying the method according to the invention, hair growth could also be observed with patients in areas free of scar tissue, i.e. in scar-free tissue. This has particularly been astonishing in areas in which no (more) hairs were growing prior to applying the method according to the invention.

When treating acute or chronic wounds, performance was as following: The method according to the invention was performed in the following way at a wound having been cleaned prior to starting the method according to the invention, wherein, e.g., fibrin plaque or necrotic tissue has been ablated by means of a sharp spoon: At first, the cell suspension and the fibrin adhesive (optional) were applied by means of a syringe or a spraying device and the areas treated were covered using a plastic dressing. The dressing was changed after about 3 to 5 days.

There is a further exemplary embodiment of a person having a ten-square centimeter area of Vitiligo on the back of the hand which had remained unchanged for years. Fifty anagen hairs were plucked out of the scalp. The hair roots separated were incubated at 37° C. for 25 min in trypsin/EDTA solution, 0.8%. Hereby, epithelial cells detached from the hair sheath. The reaction was stopped by adding human serum. The vitality of the cells measured by means of trypan blue exclusion was over 50%. The cell suspension was centrifuged at 500×g and the cellulous sediment was suspended in 2 ml of a thrombin solution. The area on the back of the hand provided for healing the skin wounds was de-epidermised by means of dermabrasio after in-depth disinfection. After applying 2 ml of a fibrin solution onto the wound area, the cell suspension was applied. Subsequently, the treatment area was occlusively covered by means of a common wound dressing. The dressing was changed every three days. After a re-epithelisation terminated within two weeks, the treatment area was illuminated twice a week by means of broad band ultraviolet beyond the erythem limit. In this way, a complete adaptation of the pigmentation of the treated skin area to the surrounding skin could be obtained in the course of eight weeks.

For suppressing an autoimmune reaction, in the case of a vitiligo, appropriate drugs that are temporary such as topical or systemic corticosteroids or topical calcineurin inhibitors are used.

1-22. (canceled)

23. A method for promoting healing of skin wounds and/or re-establishing a function of skin and/or skin appendices of a skin area, wherein the method comprises applying cells obtained from hair root sheaths of a first skin area of a donor onto a second skin area of a host.

24. The method of claim 23, wherein the cells are or comprise one or more types of precursor cells selected from melanocyte precursor cells, keratinocyte precursor cells, and mesenchymal precursor cells.

25. The method of claim 23, wherein the cells have been obtained from hair root sheaths of the first skin area by means of picking or plucking hair out of vital skin or out of a skin biopsy of the donor or have been obtained otherwise from or out of a skin biopsy of the donor.

26. The method of claim 23, wherein promoting the healing of skin wounds and/or re-establishing a function of skin and/or skin appendices of a skin area is or comprises enhancing a primary or secondary healing of wounds.

27. The method of claim 23, wherein promoting the healing of skin wounds and/or re-establishing a function of skin and/or skin appendices of a skin area is or comprises reducing or inhibiting an occurrence or development of hypertrophic scar tissue or of keloid.

28. The method of claim 23, wherein promoting the healing of skin wounds and/or re-establishing a function of skin and/or skin appendices of a skin area is or comprises enhancing or promoting a hair-growth of a scar or of skin of the skin area.

29. The method of claim 23, wherein the method further comprises a preparation of the obtained cells prior to an application thereof.

30. The method of claim 23, wherein the method further comprises applying the obtained cells without using any prior cell growth cultivation.

31. The method of claim 23, wherein the method further comprises preparing the second skin area for receiving the obtained cells.

32. The method of claim 23, wherein the method further comprises stimulating the cells after their application onto the second skin area.

33. The method of claim 23, wherein donor and host are identical.

34. A plurality of cells from hair root sheaths, wherein the cells are from hair root sheaths of a first skin area of a donor and are used in or on a second skin area of a host for promoting a healing of wounds and/or for re-establishing a function of skin and/or its appendices of the second skin area.

35. The plurality of cells of claim 34, wherein the cells are selected from one or more of melanocyte precursor cells, keratinocyte precursor cells, and mesenchymal precursor cells.

36. The plurality of cells of claim 34, wherein the cells have been obtained by picking or plucking hairs out of a vital skin or a skin biopsy of the donor or otherwise from or out of a skin biopsy of the donor.

37. The plurality of cells of claim 34, wherein the cells are obtained and provided or intended for being applied onto a second skin area of a host or for preparing a preparation, and wherein the cells are not or have not been cultivated in a cell growth cultivation prior to their application or preparation.

38. A preparation which comprises the plurality of cells of claim 34.

39. A method of preparing a preparation comprising cells from hair root sheaths, wherein the method comprises one or more of the following operations:
(i) enzymatically detaching cells from a hair root sheath;
(ii) stopping the enzymatic detachment of (i);
(iii) centrifuging or filtering a cell suspension for obtaining a sediment or a cell concentrate;
(iv) suspending the cell sediment or cell concentrate of (iii);
(v) transferring the cells of (i) or (ii) or the suspension or the sediment or the concentrate of (iii) into a biocompatible solution;
(vi) inserting or introducing the cells of (i) or (ii) or the suspension or the sediment or the concentrate of (iii) into a biocompatible support;
(vii) preparing a cell extract.

40. The method of claim 39, wherein the preparation is a suspension.

41. The method of claim 39, wherein the cells comprise one or more of melanocyte precursor cells, keratinocyte precursor cells, and mesenchymal precursor cells.

42. The method of claim 39, wherein (ii) comprises adding human serum.

43. The method of claim 39, wherein (iv) comprises suspending the cell sediment or cell concentrate of (iii) in a thrombin solution.

44. The method of claim 39, wherein the method does not comprise cultivating the cells from the hair root sheath.

45. The method of claim 39, wherein the method does not comprise (ii).

46. The method of claim 39, wherein the method does not comprise (iii).
